(19) United States
(12) Patent Application Publication
(10) Pub. No.: US 2024/0368300 A1
(43) Pub. Date: Nov. 7, 2024

(54) ANTI-CD93 ANTIBODY

(71) Applicant: UCME BIOSCIENCE S.R.L., Siena (IT)

(72) Inventors: Federico GALVAGNI, Siena (IT); Maurizio ORLANDINI, Poggibonsi (IT); Gian Marco TOSI, Siena (IT)

(21) Appl. No.: 18/682,907

(22) PCT Filed: Aug. 10, 2022
(86) PCT No.: PCT/EP2022/072408

§ 371 Date: Feb. 10, 2024

(30) Foreign Application Priority Data
Aug. 11, 2021 (IT) 102021000021764

(51) Int. Cl. C07K 16/28; G01N 33/533; G01N 33/569

(52) U.S. Cl. CPC C07K 16/2896; G01N 33/533; G01N 33/56966; C07K 2317/24; C07K 2317/565; C07K 2319/21

(57) ABSTRACT

An isolated recombinant anti-CD93 antibody for treatment of angiogenesis-related disorders is provided. The angiogenesis-related disorders are Age-related macular degeneration, diabetic retinopathies and tumors. Pharmaceutical compositions including the recombinant anti-CD93 antibody are also provided. Medical and diagnostic uses of the recombinant anti-CD93 antibody, a polynucleotide encoding the recombinant anti-CD93 antibody, vectors including the polynucleotide and host cells incorporating the vectors are further provided.

### FIELD OF THE INVENTION

The present invention concerns the field of angiogenesis-related disorders such as Age-related macular degeneration, diabetic retinopathies and tumor diseases, and in particular to compounds suitable for the treatment thereof. The invention relates to an anti-CD93 antibody, pharmaceutical compositions comprising said antibody, medical and diagnostic uses of the same. Further aspects of the invention relate to a polynucleotide encoding the anti-CD93 antibody, vectors comprising the polynucleotide and host cells incorporating said vectors.

### STATE OF THE ART

Angiogenesis is implicated in the pathogenesis of a variety of disorders, which include solid tumors, intraocular neovascular syndromes such as proliferative retinopathies or age-related macular degeneration (AMD), rheumatoid arthritis, and psoriasis [1, 2].

Monoclonal antibodies (mAbs) have emerged as a new and important pillar for cancer therapy [3]. During the past two decades molecular biology has provided means to create chimeric, humanized or fully human antibodies, also targeting angiogenesis, for the treatment of major malignant diseases and neovascular retinopathies, and to date, many antibodies and antibody-conjugates are approved as cancer, AMD and diabetic retinopathy therapeutics for marketing in Europe and the United States. They comprise unmodified antibodies, antibody-drug conjugates as well as conjugates with radionuclides and a bispecific antibody [4].

Currently, neovascular AMD (nAMD), which is characterized by choroidal neovascularization (CNV), is treated by routine intraocular injections of antibodies to vascular endothelial growth factor (VEGF), the main regulator of angiogenesis. Nevertheless, this therapy does not induce a persistent regression of the neovascularization, and frequent intravitreal injections are needed to limit the recurrence of the disorder [5]. Therefore, targeting different nAMD-associated factors represents a novel approach to develop combinatorial therapies that may allow enduring clinical benefits, avoiding the need of multiple treatments for nAMD patients. Many therapeutically active agents are only efficient within a cell. This raises a problem in case such therapeutically active agent cannot enter a cell in an unmodified form. Therefore, the need and importance is increasingly felt for binding agents, such as monoclonal antibodies or antigen-binding fragments which are efficiently internalized into tumor or endothelial cells and which can therefore target agents linked to binding agent into a malignant cell or cells of neovascular retinopathies.

Regarding the use of antiangiogenic drugs in the treatment of tumors, many studies are ongoing. In particular antiangiogenic drugs which are involved in the inhibition of the signal transduction pathways activated by members of the VEGF family, initially proved to be promising, especially in combination with chemotherapy drugs. However, this therapy has not yielded positive long-term clinical results, highlighting the need to discover new targets which allow to circumvent the mechanisms of resistance and activation of alternative angiogenesis pathways that are observed after prolonged use of monospecific antiangiogenic drugs. It is therefore object of the present invention the development of compounds able to address the above lamented problems by allowing efficient neo-angiogenesis inhibition and allowing the desired effect in the cells to be treated.

### SUMMARY OF THE INVENTION

The present invention concerns an isolated anti-CD93 (Cluster of Differentiation 93) antibody (herein also disclosed as “4E1”), in particular a recombinant antibody (herein also disclosed as “rec4E1”), which binds CD93 in a specific manner, wherein said antibody comprises:
a. a variable domain of a light chain (VJ) having the amino acid sequence of SEQ ID NO:1; and
b. a variable domain of a heavy chain (VDJ) having the amino acid sequence of SEQ ID NO:2, wherein optionally one or more conservative amino acid exchanges are present.

In a second aspect, the invention describes a polynucleotide encoding the recombinant anti-CD93 antibody.

In a further embodiment, herein described is a vector comprising the polynucleotide encoding the recombinant anti-CD93 antibody, wherein said vector is optionally an expression vector.

In a still further embodiment, the invention describes a host cell comprising the vector comprising the polynucleotide encoding the recombinant anti-CD93 antibody, wherein the host cell is preferably prokaryotic, eukaryotic, or mammalian.

A further aspect of the present invention relates to a method for producing the recombinant anti-CD93 antibody as herein described, said method comprising (a) expressing the vector comprising the polynucleotide encoding the recombinant anti-CD93 antibody in a suitable host cell, and (b) recovering the antibody.

In a further embodiment, herein described is a pharmaceutical composition comprising (i) the recombinant anti-CD93 antibody of the invention or (ii) the polynucleotide encoding the recombinant anti-CD93 antibody, wherein the composition optionally further comprises a carrier.

In a still further embodiment, the invention describes the recombinant anti-CD93 antibody of the invention or the pharmaceutical composition comprising (i) the recombinant anti-CD93 antibody of the invention or (ii) the polynucleotide encoding the recombinant anti-CD93 antibody for use as a medicament.

A further embodiment of the present invention describes the recombinant anti-CD93 antibody of the invention or the pharmaceutical composition comprising (i) the recombinant anti-CD93 antibody of the invention or (ii) the polynucleotide encoding the recombinant anti-CD93 antibody for use in the treatment of: neovascular retinopathies, preferably wherein the retinopathy is AMD and diabetic retinopathy, intraocular neovascular syndromes, proliferative retinopathies, rheumatoid arthritis, psoriasis, and/or a tumor disease, preferably wherein the tumor disease is an epithelial tumor disease and/or is selected from the group consisting of astrocytoma, oligodendroglioma, meningioma, neurofibroma, glioblastoma, ependymoma, Schwannoma, neurofibrosarcoma, medulloblastoma, melanoma, pancreatic cancer, cancer, endometrial cancer, renal cancer, neuroblastoma, squamous carcinoma, hepatoma, colon cancer, mesothelioma and epidermoid carcinoma.

A further object of the invention relates to the recombinant anti-CD93 antibody as herein described, wherein said antibody is linked to a diagnostic compound, preferably selected from a radionuclide, a chemoluminescent compound, a fluorescent compound, a dye or an enzyme.

A further object of the invention relates to the use of the rec4E1 antibody as herein described, both linked or not linked to a diagnostic compound, as an in vitro diagnostic agent or as an in vitro biotechnological agent.

The problem underlying the present invention is that of making available compounds capable of treating angiogenesis-related disorders such as AMD.

This problem is solved by the present finding by the development of compounds capable of blocking neo-angiogenesis as described in the attached claims.

### BRIEF DESCRIPTION OF THE DRAWINGS

The characteristics and advantages of the present invention will be apparent from the detailed description reported below, from the Examples given for illustrative and non-limiting purposes, and from the annexed FIGS. 1-5 wherein:

FIG. 1. CD93 and Multimerin-2 (MMRN2) are coexpressed in blood vessels within human tumors.

A: Correlation analysis of CD93 and MMRN2 or PECAM-1 RNA-seq data collected from different human cancer types. Data were generated by The Cancer Genome Atlas and are presented in a scatter plot as average of FPKM (number Fragments Per Kilobase of exon per Million reads).

B: Immunoistochemical staining of CD93 and MMRN2 in representative human cancer types: renal, high staining in blood vessel, and cervical, low staining in blood vessels. Magnification of blood vessels is shown.

C: Semiquantitative scoring of the fraction of positive blood vessels in human cancer tissue microarrays. Values are reported as scoring average. The number of examined samples for each cancer type (CD93 and MMRN2 respectively) was: thyroid, 7-4; lung, 23-11; liver, 22-12; pancreatic, 19-11; head and neck, 8-3; stomach, 22-11; colorectal, 24-10; renal, 23-12; prostate, 16-12; testis, 23-10; breast, 23-11; cervical, 24-12; ovarian, 22-12; endometrial, 22-12; melanoma, 23-11.

D: Correlation analysis of semiquantitative scoring of the fraction of CD93- and MMRN2-positive blood vessels in cancer tissue microarrays analyzed as in C. The correlation coefficient (r) was obtained using linear regression (Pearson's) analysis. P (two-tailed) values are indicated.

FIG. 2. The anti-CD93 4E1 mAb is internalized via the lysosomal pathway.

Human umbilical vein endothelial cells (HUVECs) were incubated 18 h with 4E1 in the presence or not of 100 nM Bafilomycin A1. Then cells were fixed and stained with Alexa Fluor 488 labeled anti-mouse IgG and ToPro-3 to label nuclei. Bafilomycin A1 is a specific inhibitor of vacuolar-type proton pump, which inhibits acidification and protein degradation in lysosomes and promotes perinuclear distribution of lysosomes. After Bafilomycin A1 treatment 4E1 staining shows perinuclear accumulation.

FIG. 3. The anti-CD93 4E1 mAb is internalized via the endosomal-lysosomal pathway.

HUVECs were incubated 18 h with 4E1 in the presence or not of 100 nM Bafilomycin A1. Then cells were fixed, incubated with a rabbit anti-Rab7 antibody and then stained with Alexa Fluor 488 labeled anti-mouse IgG and Alexa Fluor 568 labeled anti-rabbit IgG. After Bafilomycin A1 treatment, 4E1 shows co-staining with Rab7, a known marker of late endosomes that controls aggregation and fusion of late endocytic structures/lysosomes [6].

FIG. 4. Biacore analysis of CD93-4E1 binding and affinity. (a) Sensorgram of CD93 binding to surface immobilized 4E1. Different concentrations of CD93 were injected over 4E1, previously immobilized on a SA sensor chip. (b) Kinetics constants and affinity values of CD93 binding to 4E1 mAb calculated applying a 1:1 binding as fitting model using the Bia T200 evaluation software 2.0.1.

FIG. 5. Conjugated 4E1 colocalizes with MMRN2 in endocytic vesicles.

HUVECs at late phases of spreading were stained using Alexa Fluor 488-labeled 4E1 and a polyclonal anti-MMRN2 antibody, which was revealed by Alexa Fluor 568-conjugated secondary antibody. Merged and differential interference contrast (DIC) images are shown.

FIG. 6. Purification of rec4E1. The chromatogram shows the Protein A-affinity purification of rec4E1 from cell supernatant harvested at different days of culture. A step-gradient elution protocol based on sodium citrate pH 3 (50, 75, and 100%) was used. The y-axis represents the UV reading expressed as arbitrary units (AU) and the x-axis the volume of fractions.

FIG. 7. Quality control of purified rec4E1. A: Gel showing the SDS-PAGE (8%) analysis of the rec4E1 pooled fractions loaded at different concentrations. Increasing antibody concentrations revealed the absence of non-specific proteins. The gel was stained with Coomassie blue. B: Western blot analysis of the pooled fractions using a HRP-conjugated anti-mouse antibody. Supernatant from non-transfected CHO cells was used as negative control (Ctrl).

FIG. 8. Rec4E1 recognizes the conformational epitope of the human CD93 protein expressed in human cells. Human 293 cells were transiently transfected with a construct expressing human full-length CD93. Cell extracts were analyzed by Western blot under non-reducing conditions using purified rec4E1 or 4E1 antibodies. Supernatant from non-transfected CHO cells was used as a negative control (Ctrl).

FIG. 9. Biacore analysis of CD93-rec4E1 binding and affinity. (a) Sensorgram, expressed in resonance units (RU), of CD93 binding to surface immobilized rec4E1. Different concentrations of CD93 were injected over rec4E1, previously immobilized on a SA sensor chip. (b) Kinetics constants and affinity values of CD93 binding to rec4E1 mAb calculated applying a 1:1 binding as fitting model using the Bia T200 evaluation software 2.0.1.

FIG. 10. Rec4E1 affects endothelial cell behavior. Representative images of a Matrigel tube formation assay. HUVECs were seeded on antibody-containing Matrigel and challenged with purified antibodies as indicated. Images were taken following 8 h of incubation.

### DETAILED DESCRIPTION OF THE INVENTION

The present invention concerns an isolated anti-CD93, preferably a recombinant anti-CD93 monoclonal antibody named rec4E1, wherein said antibody comprises:
a. a variable domain of a light chain (VJ) having the amino acid sequence of SEQ ID NO:1; and
b. a variable domain of a heavy chain (VDJ) having the amino acid sequence of SEQ ID NO:2, wherein optionally one or more conservative amino acid exchanges are present.

CD93 is a protein that in humans is encoded by the CD93 gene. CD93 is a C-type lectin transmembrane receptor expressed by a wide variety of cells, which plays a role not only in cell-cell and cell-extracellular matrix adhesion processes but also in angiogenesis, both as a soluble growth factor and as adhesion molecule. The Genbank entry for human CD93 protein is NP_036204 and human CD93 cDNA sequence is NM_012072. The Genbank entry for the murine CD93 protein is NP_034870 and murine CD93 cDNA sequence is NM_010740.

Results from Matrigel plug assays in mice and in vitro angionesis assay in human primary endothelial cells have suggested that the mAb 4E1 is a promising tool for cancer therapy. This mAb (isotype IgG1 k light chain) recognizes a conformational epitope on CD93 molecule in a region shared between the C type lectin-like domain (CTLD) and sushi domain, and could be a good base for the generation of Antibody-Drug Conjugates (ADCs), because in endothelial cells it is internalized via endocytosis into the lysosomal pathway, a necessary step which results in the degradation of ADC due to the numerous proteolytic enzymes present in the acidic lysosomal compartment with subsequent release of the drug payload (FIGS. 2 and 3). The variable regions of mAb 4E1 were sequenced and cloned into a plasmid vector specifically designed to produce whole recombinant antibodies of the IgG1 isotype in high yield (Example 5). The obtained expression plasmid was successfully used for the expression and purification of the recombinant 4E1 antibody (rec4E1) from the supernatant of CHO cells (FIGS. 6 and 7). The purified rec4E1 retains the binding specificity of mAb 4E1 for the conformational epitope of CD93 expressed by human cells (FIG. 8). Moreover, rec4E1 shows higher affinity for CD93 than mAb 4E1 (FIGS. 4 and 9). Finally, rec4E1 retains the inhibitory effect of mAb 4E1 in an in vitro angiogenesis assay (FIG. 10).

In a preferred aspect, the anti-CD93 recombinant 4E1 antibody of the present invention comprises 6 CDR regions, said CDR regions being:
a. a CDR-L1 having the amino acid sequence of SEQ ID NO:5;
b. a CDR-L2 having the amino acid sequence of SEQ ID NO:6;
C. a CDR-L3 having the amino acid sequence of SEQ ID NO:7;
d. a CDR-H1 having the amino acid sequence of SEQ ID NO:8;
e. a CDR-H2 having the amino acid sequence of SEQ ID NO:9; and
f. a CDR-H3 having the amino acid sequence of SEQ ID NO:10, wherein optionally one or more conservative amino acid exchanges are present, whereas by conservative amino acid exchanges or replacement, it is meant an exchange of an amino acid into another that has similar properties. This type of exchange is expected to rarely result in dysfunction in the corresponding protein. Similarity between amino acids can be calculated based on substitution matrices, physico-chemical distance, or simple properties such as amino acid size or charge.

These anti-CD93 antibodies which have conservative amino acid exchanges in the six CDR regions are all capable of binding to the same CD93 epitope recognized by the mAb rec4E1.

An epitope is the part of an antigen that is recognized by antibodies or related anti-CD93 antibodies. For example, the epitope is the specific piece of the antigen that an antibody binds to. Epitopes may be conformational epitopes or linear epitopes. A conformational epitope is composed of discontinuous sections of the antigen's amino acid sequence. These epitopes interact with the paratope based on the 3-D surface features and shape or tertiary structure of the antigen. The proportion of epitopes that are conformational is unknown.

It was shown that 4E1 and rec4E1 bind to and recognize an epitope on CD93 molecule within the CTLD and sushi domains of CD93. Methods for determining an epitope bound and recognized by a binding molecule are described in the prior art. For fine mapping, recombinant Myc-tagged CD93 fragments can be used, as for example as described in [7]. The recombinant proteins can be used in Immunoprecipitation and Western blot analyses for epitope mapping. The mAb 4E1 and rec4E1 react with the CTLD and sushi domains of CD93. In general, methods for determining the epitope of a given antibody are known in the art and include the preparation of synthetic linear peptides of a given region of interest and the subsequent testing whether the antibody binds to said peptides [8]. Alternatively, different recombinant proteins covering the region of interest can be produced and tested for the binding of the antibody.

As described in [7], the epitope of the mAb 4E1 is within the CTLD and sushi domains of CD93. Therefore, also the epitope of the binding molecules, in particular monoclonal antibodies or antigen-binding fragments thereof of the invention is preferably within CTLD and sushi domains of CD93.

In a preferred embodiment, the epitope is within the CTLD and sushi domains of CD93.

The mAb 4E1 and rec4E1 have the following CDR sequences: KASQSVDYDGDSYMN (LCDR1; SEQ ID No: 5), AASNLES (LCDR2; SEQ ID No: 6), QQNNEDPRT (LCDR3; SEQ ID No: 7), GYTFASY (HCDR1; SEQ ID No: 8), YPGNGD (HCDR2; SEQ ID No: 9), and LDWYFDL (HCDR3; SEQ ID No: 10).

The above-mentioned sequences show the CDRs of the monoclonal antibody 4E1 and rec4E1 determined according to the method of Chothia, which is generally known in the art.

For the purposes of the present disclosure, each sequence has a corresponding SEQ ID NO. as follows:
SEQ ID No: 1 relates to the VJ domain, i.e. the variable part, of the light chain of 4E1 and rec4E1: DIVLTQSPTSLAVSLGQRATISCKASQSVDYDGDSYMNWYQQKPGQPPRLLIYAAS NLESGIPARFSGSGSGTDFTLNIHPVEEEDAATYYCQQNNEDPRTFGGGTKLEIK
SEQ ID No: 2 relates to the VDJ domain, i.e. the variable part of the heavy chain of 4E1 and rec4E1: QEQLQQPGTELVKPGASVRLSCKTSGYTFASYTLHWVKQTPGQGLDWIGAIYPGN GDTSYNQKFKDKATLTADKSSSTAYMQLSNLTSEDSAVYYCARLDWYFDLWGAGT TVTVS
SEQ ID NO:3 corresponds to the cDNA sequence of the immunoglobulin gene of the 4E1 and rec4E1 monoclonal antibody light chain (cDNA of leader peptide in lowercase, cDNA of SEQ ID No: 1 in uppercase): atggagtttcagacccaatcctgctatgggtgctcctgctctgggttccaggctccactggtGACATTGTGCTGA CCCAATCTCCAACTTCTTTGGCTGTGTCTCTAGGGCAGAGGGCCACCATCTCCT GCAAGGCCAGCCAAAGTGTTGATTATGATGGTGATAGTTATATGAACTGGTATC AACAGAAACCAGGACAGCCACCCAGACTCCTCATCTATGCTGCATCCAATCTAG AATCTGGGATCCCAGCCAGGTTTAGTGGCAGTGGGTCTGGGACAGACTTCACC CTCAACATCCATCCTGTGGAGGAGGAGGATGCTGCAACCTATTACTGTCAGCAA AATAATGAGGATCCTCGGACGTTCGGTGGAGGCACCAAGTTGGAAATAAAAC
SEQ ID NO:4 corresponds to the cDNA sequence of the immunoglobulin gene of the 4E1 monoclonal antibody heavy chain (cDNA of leader peptide in lowercase, cDNA of SEQ ID No: 2 in uppercase): atgggatggagctgtatcatcctcttcttggtagcaacagctacaggtgtccactccCAGGAGCAACTGCAG CAGCCTGGGACTGAGCTGGTGAAGCCTGGGGCCTCAGTGAGGCTGTCCTGCAA GACTTCTGGCTACACATTTGCCAGTTACACTTTGCACTGGGTAAAGCAGACACC TGGACAGGGCCTGGACTGGATTGGAGCTATTTATCCAGGAAATGGTGATACTTC CTACAATCAGAAATTCAAAGACAAGGCCACATTAACTGCAGACAAATCTTCCAGC ACAGCCTATATGCAGCTCAGTAACCTGACATCTGAGGACTCTGCGGTCTATTAC TGTGCAAGGTTAGACTGGTACTTCGATCTCTGGGGCGCAGGGACCACGGTCAC TGTCTCTGCAG

The sequences of the CDRs of the light chain (VJ domain) of 4E1 and rec4E1 according to Chothia are as follows:
SEQ ID NO:5 corresponds to the amino acid sequence of the CDR-L1 (or LCDR1): KASQSVDYDGDSYMN
SEQ ID NO:6 corresponds to the amino acid sequence of the CDR-L2 (or LCDR2): AASNLES
SEQ ID NO:7 corresponds to the amino acid sequence of the CDR-L3 (or LCDR3): QQNNEDPRT

The sequences of the CDRs of the heavy chain (VDJ domain) of 4E1 and rec4E1 according to Chothia are as follows:
SEQ ID NO:8 corresponds to the amino acid sequence of the CDR-H1 (or HCDR1): GYTFASY
SEQ ID NO:9 corresponds to the amino acid sequence of the CDR-H2 (or HCDR2): YPGNGD
SEQ ID NO:10 corresponds to the amino acid sequence of the CDR-H3 (or HCDR3): LDWYFDL.

In a further embodiment, the present invention relates to an anti-CD93 antibody which competes with the mAb rec4E1 for binding to CD93, wherein the variable part of the light chain of rec4E1 comprises, preferably has the sequence according to SEQ ID No: 1 or wherein the light chain is encoded by SEQ ID No: 3, and wherein the heavy chain of rec4E1 comprises, preferably has the sequence according to SEQ ID No: 2 or wherein the heavy chain is encoded by SEQ ID No: 4.

Competition may be determined by assays known to a skilled person, such as Competition binding assays.

In a still preferred aspect, the isolated anti-CD93 and rec4E1 antibody is a monoclonal antibody, and/or is humanized or human.

Similar to tumor blood vessels, endothelial cells of choroidal neovessels from nAMD patients express high levels of CD93. In endothelial cells, the interaction between CD93 and MMRN2 activates signaling pathways essential for vascular development and angiogenesis. It has been demonstrated that, similarly to what observed in hyperproliferative endothelial cells of tumor blood vessels (as will be shown in the Examples), CD93 and MMRN2 are highly expressed in the choroidal vasculature from nAMD patients [9]. Using an ex vivo model of microvascular angiogenesis, it was shown that both choroidal tissues isolated from CD93 knockout mice and human choroidal tissue cultured in the presence of the CD93 neutralizing antibody rec4E1 produced a reduced vascular sprouting in comparison to control choroidal tissues. Moreover, the experimental in vivo model of laser-induced CNV, which recapitulates the main features of the exudative form of AMD, shows a reduced neovascularization in CD93 knockout mice. Altogether, these results point out the major role of CD93-MMRN2 interaction in vascular physiology of the choroid, opening up new possibilities to therapeutic intervention to CNV.

WO2020180706A1 discloses the anti-CD93 antibody F11 which binds to a undefined epitope within CD93. In comparison to F11, rec4E1 has the peculiar features of blocking angiogenesis by itself, by inhibiting the interaction of CD93 with its ligand MMRN2 [10].

In a still preferred aspect, the recombinant anti-CD93 antibody is a diabody, triabody or tetrabody, and more preferably the antibody is a full-length monoclonal antibody and is optionally a bispecific antibody.

It is preferred that the anti-CD93 antibody of the Invention exhibits a strong affinity to CD93. As will be shown in the Examples, 4E1 and rec4E1 exhibit an affinity of KD (M)=4.50*10⁻¹⁰ and 5.09*10⁻¹² for CD93, respectively (Example 3 and Example 7).

Affinity to CD93 may be determined by methods known in the art, as for example by surface plasmon resonance. The binding analysis for 4E1 and rec4E1 was performed using a BIAcore 3000 equipped with a CM5 sensor chip. Briefly, a BIAcore CM5 chip was activated with EDC/NHS and 4E1 or rec4E1 mAbs were captured onto the activated surface. The remaining active sites were blocked by ethanolamine/HCl. Recombinant CD93 was bound to the 4E1 or rec4E1 surface and allowed to dissociate over time. The association and dissociation phases for each injection over each density surface were subjected to kinetic analysis.

Therefore, in a yet further preferred embodiment, the recombinant anti-CD93 antibody of the invention, preferably monoclonal antibody or antigen-binding fragment thereof, binds CD93 with an affinity (KD) of at least 10⁻¹⁰, preferably of at least 10⁻¹¹, more preferably of at least 10⁻¹².

In a second aspect, the invention describes a polynucleotide encoding the anti-CD93 antibody. The polynucleotide encodes a polypeptide, which exhibits specific binding to the CD93 target. “Specific binding” is understood to mean that the binding of the anti-CD93 antibody to CD93 is at least 50-fold, preferably at least 100-fold stronger than the binding to a control protein such as albumin, as determined e.g. by Western Blot analysis or ELISA.

The term “anti-CD93 antibody”, as used herein, means any polypeptide which has structural similarity to a naturally occurring antibody and is capable of binding to CD93, wherein the binding specificity is determined by the CDRs of the polypeptides. Hence, “anti-CD93 antibody” is intended to relate to an immunoglobulin-derived structure with binding to CD93. The antigen-binding fragment is understood as polypeptide, which comprises at least one antigen binding fragment of a full-length antibody. Antigen binding fragments consist of at least the variable domain of the heavy chain and the variable domain of the light chain, arranged in a manner that both domains together are able to bind to the specific antigen.

The term “recombinant”, as used herein, means are antibody fragments produced by using molecular biology technologies and have many advantages in both medical and research applications. Advantages of recombinant antibodies are that they are stable over time, they do not undergo unwanted changes which may affect its functionality and maintain high standard of specificity.

Monoclonal antibodies are monospecific antibodies that are identical because they are produced by one type of immune cell (clone) originated from a single parent cell. “Monoclonal antibodies” and the production of monoclonal antibodies belong to the state of the art. In general, monoclonal antibodies can, for example, be prepared in accordance with the known method of Winter & Milstein [11]. An alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest.

In a further embodiment, herein described is a vector comprising the polynucleotide encoding the recombinant anti-CD93 antibody, wherein said vector is optionally an expression vector.

Such vector is preferably a vector for expression in bacteria, such as E. coli, in a yeast cell, in insect cells or mammalian cells. Preferably, the nucleic acids of the invention are under the control of suitable regulatory sequences such as promoters or enhancers in the vector, thereby allowing expression in a host cell. The vectors preferably comprise sequences enabling replication in a host cell.

In a still further embodiment, the invention describes a host cell comprising the vector comprising the polynucleotide encoding the recombinant anti-CD93 antibody, wherein the host cell is preferably prokaryotic, eukaryotic, or mammalian.

A further aspect of the present invention relates to a method for producing the anti-CD93 antibody as herein described, said method comprising (a) expressing the vector comprising the polynucleotide encoding the recombinant anti-CD93 antibody in a suitable host cell, and (b) recovering the antibody.

In a further embodiment, herein described is a pharmaceutical composition comprising (i) the recombinant anti-CD93 antibody of the invention or (ii) the polynucleotide encoding the recombinant anti-CD93 antibody, wherein the composition optionally further comprises a carrier.

Preferably in the pharmaceutical composition according to the invention said anti-CD93 antibody is linked to a therapeutically active substance, preferably to a chemotherapeutic compound, preferably selected from an alkylating agent, antineoplastic antibiotic, antimetabolite, and a natural source derivative, a cytotoxic compound, a cytostatic compound, a cytokine, a nanoparticle, or a radionuclide.

It was found that the mAb 4E1 exhibits an efficient internalization into CD93-bearing mammalian cells, as determined by microscopic analysis of such cells incubated with 4E1 as shown in the Examples.

Therefore, the monoclonal antibody according to the present invention and other anti-CD93 antibodies, which bind to CD93 in the same epitope of CD93 as recognized by 4E1, are surprisingly advantageous in the field of biotechnological research, diagnosis or therapy. In particular, an active agent may be linked to an anti-CD93 antibody of the invention, which is taken up into the cell by internalization. Such active agent may then exert the desired effect in the cell, such as a cytotoxic or cytostatic effect.

In a preferred embodiment, the therapeutically active substance is a chemotherapeutic compound or a cytotoxic compound or a cytostatic compound selected from a DNA damaging agent, in particular actinomycin-D, mitomycin C, cisplatin, doxorubicin, etoposide, verapamil, podophyllotoxin, 5-FU, a natural source derivative and a taxan, preferably paclitaxel and carboplatin, or more preferably wherein the anti-CD93 antibody is covalently linked to a therapeutically active substance of (a) and/or the diagnostic compound of (b), optionally via a linker.

In a preferred aspect, the pharmaceutical composition of the invention is for oral, or parenteral, topical, rectal, intravenous, subcutaneous, intramuscular, intranasal, intravaginal, through the oral mucosa, the lung mucosa, or for transocular administration, wherein said pharmaceutical composition is administered incorporated into liposomes, microvescicles, bound to molecular carriers or combined with molecules selected from the group consisting of molecules that allow the temporary opening of the blood-brain barrier, anti-inflammatory molecules, monoclonal antibodies and drugs with immunosuppressive activity.

In a still further embodiment, the invention describes the recombinant anti-CD93 antibody of the invention or the pharmaceutical composition comprising (i) the recombinant anti-CD93 antibody of the invention or (ii) the polynucleotide encoding the recombinant anti-CD93 antibody for use as a medicament.

The unique properties of rec4E1 allow improved and accelerated drug delivery to endothelial cells associated to cancer or neovascular retinopathies.

A further embodiment of the present invention describes the recombinant anti-CD93 antibody of the invention or the pharmaceutical composition comprising (i) the recombinant anti-CD93 antibody of the invention or (ii) the polynucleotide encoding the recombinant anti-CD93 antibody for use in the treatment of, neo-angiongenic retinopathies, preferably wherein the retinopathy is AMD and diabetic retinopathy, intraocular neovascular syndromes, proliferative retinopathies, rheumatoid arthritis, psoriasis, and/or a tumor disease, preferably wherein the tumor disease is an epithelial tumor disease and/or is selected from the group consisting of astrocytoma, oligodendroglioma, meningioma, neurofibroma, glioblastoma, ependymoma, Schwannoma, neurofibrosarcoma, medulloblastoma, melanoma, pancreatic cancer, cancer, endometrial cancer, renal cancer, neuroblastoma, squamous carcinoma, hepatoma, colon cancer, mesothelioma and epidermoid carcinoma.

A further object of the invention relates to the recombinant anti-CD93 antibody as herein described, wherein said antibody is linked to a diagnostic compound, preferably selected from a radionuclide, a chemoluminescent compound, a fluorescent compound, a dye or an enzyme.

Preferably, the anti-CD93 antibody linked to a diagnostic compound may be used for diagnostic purposes in vitro and thereby relates to an important tool for biotechnological research. For example, biopsies of patients or bodily samples in general may be analyzed using the anti-CD93 and recombinant anti-CD93 antibodies of the invention. Therefore, a further embodiment, the present invention relates to the use of an anti-CD93 antibody of the invention as an in vitro diagnostic agent or as an in vitro biotechnological agent. In a more preferred embodiment, the present invention relates to the use of an anti-CD93 antibody of the invention linked to a diagnostic compound as an in vitro diagnostic agent or as an in vitro biotechnological agent.

It was found that 4E1 is efficiently internalized into human primary endothelial cells, as shown in the Examples and FIGS. 2 and 3. Internalization may be determined using an anti-CD93 antibody which is linked to a suitable diagnostic compound such as a fluorescent compound. In the examples, Alexa488 was used as fluorescent dye. The quantification of internalization may be either performed by microscopic analysis, as described in the example, or by imaging flow cytometry. It was found that 4E1 was internalized in late endosomes/lysosomes, and therefore, 4E1 or antigen-binding fragments thereof or anti-CD93 antibodies recognizing the same epitope as 4E1, are in particular suitable for delivery of a therapeutically active agent (drug delivery) or for delivery of a diagnostic compound, e.g. for imaging purposes.

The anti-CD93 antibody of the invention can be internalized by a mammalian cell expressing CD93, preferably said mammalian cell is an endothelial cell or macrophage. In a preferred embodiment, internalization by the mammalian cell expressing CD93, preferably wherein the cell is an endothelial cell or macrophage, is determined by microscopic analysis and quantification, or by imaging flow cytometry. In a particularly preferred embodiment, the anti-CD93 antibody of the invention is linked to a therapeutically active substance, which allows internalization of the therapeutically active substance. A therapeutically active substance according to the invention is understood as a compound, which has a therapeutic or preventive effect in an animal, preferably mammal, even more preferably human. The therapeutically active substance can be a compound useful in chemotherapy, i.e. a chemotherapeutic compound. In a preferred embodiment, the therapeutically active substance is a cytotoxic or cytostatic compound.

Therefore, in one embodiment, the invention relates to an anti-CD93 antibody of the invention linked to a therapeutically active substance, preferably to a chemotherapeutic compound, preferably selected from an alkylating agent, antineoplastic antibiotic, antimetabolite, and a natural source derivative, a cytotoxic compound, a cytostatic compound, a cytokine, a nanoparticle, or a radionuclide. Suitable cytokines are for example TNFalpha, IL-2 and IL-12.

Suitable chemotherapeutic compounds are known in the art. These compounds fall into several different categories, including, for example, alkylating agents, antineoplastic antibiotics, antimetabolites, and natural source derivatives.

Examples of alkylating agents that can be used in the invention include busulfan, caroplatin, carmustine, chlorambucil, cisplatin, cyclophosphamide (i.e., Cytoxan), dacarbazine, ifosfamide, lomustine, mechlorethamine, melphalan, procarbazine, streptozocin, and thiotepa.

Examples of antineoplastic antibiotics include bleomycin, dactinomycin, daunorubicin, doxorubicin, idarubicin, mitomycin (e.g., mitomycin C), mitoxantrone, pentostatin, and plicamycin.

Examples of antimetabolites include fluorodeoxyuridine, cladribine, cytarabine, floxuridine, fludarabine, flurouracil (e.g., 5-fluorouracil (5FU)), gemcitabine, hydroxyurea, mercaptopurine, methotrexate, and thioguanine.

Examples of natural source derivatives include docetaxel, etoposide, irinotecan, taxanes (e.g. paclitaxel), teniposide, topotecan, vinblastine, vincristine, vinorelbine, prednisone, and tamoxifen.

Additional examples of chemotherapeutic agents that can be used in the invention include asparaginase and mitotane.

Furthermore, also C2 ceramide can be used.

Further, it may be advantageous to link the anti-CD93 antibodies of the invention to a diagnostic compound. A diagnostic compound is a compound, which is capable of producing a signal via direct or indirect detection. Further, it may be advantageous to link the anti-CD93 antibodies of the invention to a diagnostic compound.

A diagnostic compound is a compound, which is capable of producing a signal via direct or indirect detection.

“Full length” or “complete” antibodies refer to proteins that comprise two heavy (H) and two light (L) chains inter-connected by disulfide bonds which comprise: (1) in terms of the heavy chains, a variable region and a heavy chain constant region which comprises three domains, C_{H}1, C_{H}2 and C_{H}3; and (2) in terms of the light chains, a light chain variable region and a light chain constant region which comprises one domain, CL. With regard to the term “complete antibody”, any antibody is meant that has a typical overall domain structure of a naturally occurring antibody (i.e. comprising a heavy chain of three or four constant domains and a light chain of one constant domain as well as the respective variable domains), even though each domain may comprise further modifications, such as mutations, deletions, or insertions, which do not change the overall domain structure. For instance, the mAb 4E1 or rec4E1 is a full-length antibody.

An “antigen-binding fragment” of a monoclonal antibody is a fragment of a monoclonal antibody, which exhibits essentially the same function and specificity as the complete monoclonal antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

As the first generation of full-sized antibodies presented some problems, many of the second-generation antibodies have comprised only fragments of the antibody. Variable domains (Fvs) are the smallest fragments with an intact antigen-binding domain consisting of one V_{L} and one V_{H}. Such fragments, with only the binding domains, can be generated by enzymatic approaches or expression of the relevant gene fragments, e.g. in bacterial and eukaryotic cells. Different approaches can be used, e.g. either the Fv fragment alone or ‘Fab’-fragments comprising one of the upper arms of the “Y” that includes the Fv plus the first constant domains. These fragments are usually stabilized by introducing a polypeptide link between the two chains, which results in the production of a single chain Fv (scFv). Alternatively, disulfide-linked Fv (dsFv) fragments may be used. The binding domains of fragments can be combined with any constant domain in order to produce full length antibodies or can be fused with other proteins and polypeptides.

A recombinant antibody fragment is the single-chain Fv (scFv) fragment. In general, it has a high affinity for its antigen and can be expressed in a variety of hosts. These and other properties make scFv fragments not only applicable in medicine, but also of potential for biotechnological applications. As detailed above, in the scFv fragment the V_{H} and V_{L} domains are joined with a hydrophilic and flexible peptide linker, which improves expression and folding efficiency. Usually, linkers of about 15 amino acids are used, of which the (Gly₄Ser)₃ linker has been used most frequently. scFv molecules might be easily proteolytically degraded, depending on the linker used. With the development of genetic engineering techniques these limitations could be practically overcome by research focused on improvement of function and stability. An example is the generation of disulfide-stabilized (or disulfide-linked) Fv fragments where the V_{H}-V_{L} dimer is stabilized by an interchain disulfide bond. Cysteines are introduced at the interface between the V_{L} and V_{H} domains, forming a disulfide bridge, which holds the two domains together.

Dissociation of scFvs results in monomeric scFvs, which can be complexed into dimers (diabodies), trimers (triabodies) or larger aggregates such as TandAbs and Flexibodies.

Antibodies with two binding domains can be created either through the binding of two scFv with a simple polypeptide link (scFv) 2 or through the dimerization of two monomers (diabodies). The simplest designs are diabodies that have two functional antigen-binding domains that can be either the same, similar (bivalent diabodies) or have specificity for distinct antigens (bispecific diabodies).

Also, antibody formats comprising four variable domains of heavy chains and four variable domains of light chains have been developed. Examples of these include tetravalent bispecific antibodies (TandAbs and Flexibodies, Affimed Therapeutics AG, Heidelberg. Germany). In contrast to a bispecific diabody, a bispecific TandAb is a homodimer consisting of only one polypeptide. Because the two different chains, a diabody can build three different dimers only one of which is functional. Therefore, it is simpler and cheaper to produce and purify this homogeneous product. Moreover, the TandAb usually shows better binding properties (possessing twice the number of binding sites) and increased stability in vivo. Flexibodies are a combination of scFv with a diabody multimer motif resulting in a multivalent molecule with a high degree of flexibility for joining two molecules which are quite distant from each other on the cell surface. If more than two functional antigen-binding domains are present and if they have specificity for distinct antigens, the antibody is multispecific.

In summary, specific immunoglobulins, into which particular disclosed sequences may be inserted or, in the alternative, form the essential part of, include but are not limited to the following anti-CD93 antibodies which form particular embodiments of the present invention: a Fab (monovalent fragment with variable light (V_{L}), variable heavy (V_{H}), constant light and constant heavy 1 (CHI) domains), a F(ab′)2 (bivalent fragment comprising two Fab fragments linked by a disulfide bridge or alternative at the hinge region), a Fv (V_{L} and V_{H} domains), a scFv (a single chain Fv where V_{L} and V_{H} are joined by a linker, e.g., a peptide linker), a bispecific antibody molecule (an antibody molecule comprising a polypeptide as disclosed herein linked to a second functional moiety having a different binding specificity than the antibody, including, without limitation, another peptide or protein such as an antibody, or receptor ligand), a bispecific single chain Fv dimer, a diabody, a triabody, a tetrabody, a minibody (a scFv joined to a CH₃).

Certain anti-CD93 antibodies or antigen-binding fragments of monoclonal antibodies including, but not limited to, Fv, scFv, diabody molecules or domain antibodies (Domantis) may be stabilized by incorporating disulfide bridges to line the V_{H} and V_{L} domains. Bispecific antibodies may be produced using conventional technologies, specific methods of which include production chemically, or from hybridomas and other technologies including, but not limited to, the BiTE™ technology (molecules possessing antigen binding regions of different specificity with a peptide linker) and knobs-into-holes engineering.

Accordingly, the anti-CD93 antibody or antigen-binding fragment of a monoclonal antibody may be a Fab, a Fab′, a F(ab′)2, a Fv, a disulfide-linked Fv, a scFv, a (scFv) 2, a bivalent antibody, a bispecific antibody, a multispecific antibody, a diabody, a triabody, a tetrabody or a minibody.

In another preferred embodiment, the recombinant anti-CD93 antibody is a human antibody, chimeric antibody or a humanized antibody. A chimeric antibody is an antibody in which at least one region of an immunoglobulin of one species is fused to another region of an immunoglobulin of another species by genetic engineering in order to reduce its immunogenecity. For example, murine V_{L} and V_{H} regions may be fused to the remaining part of a human immunoglobulin. A particular type of chimeric antibodies are humanized antibodies. Humanized antibodies are produced by merging the DNA that encodes the CDRs of a non-human antibody with human antibody-producing DNA. The resulting DNA construct can then be used to express and produce antibodies that are usually not as immunogenic as the non-human parenteral antibody or as a chimeric antibody, since merely the CDRs are non-human. Further, the antibody may be human.

A further object of the invention relates to the use of the anti-CD93 antibody as herein described, both linked or not linked to a diagnostic compound, as an in vitro diagnostic agent or as an in vitro biotechnological agent.

Various embodiments and aspects of the present invention as delineated hereinabove and as claimed in the claims section below find experimental support in the following examples.

### Examples

Reference is now made to the following examples, which together with the above descriptions illustrate some embodiments of the invention.

### Example 1: CD93 and MMRN2 Expression Correlation

### Materials and Methods

### RNA-Seq Data, Tissue Microarray, and Image Analysis

The CD93, MMRN2, and PECAM-1 RNA-seq data from 15 diverse cancer types were generated by The Cancer Genome Atlas (Table 1).

TABLE 1
RNA-seq data of CD93, MMRN2, and PECAM-1
in different cancer types.

| | Average FPKM | | | n |
|---|---|---|---|---|
| cancer type | CD93 | MMRN2 | PECAM-1 | samples |
| Thyroid | 12.7 | 9.3 | 25.1 | 501 |
| Lung | 11.2 | 3.9 | 20.6 | 994 |
| Liver | 5.9 | 4.1 | 11.1 | 365 |
| Pancreatic | 17.0 | 6.5 | 26.7 | 176 |
| Head and neck | 7.5 | 4.0 | 11.6 | 499 |
| Stomach | 13.6 | 6.0 | 22.9 | 354 |
| Colorectal | 7.6 | 2.7 | 11.3 | 597 |
| Renal | 29.7 | 15.9 | 59.9 | 877 |
| Prostate | 7.0 | 4.7 | 9.4 | 494 |
| Testis | 6.3 | 3.7 | 12.6 | 134 |
| Breast | 14.8 | 6.7 | 22.3 | 1075 |
| Cervical | 3.9 | 2.6 | 7.8 | 291 |
| Ovarian | 4.1 | 5.0 | 6.4 | 373 |
| Endometrial | 5.5 | 3.6 | 10.0 | 541 |
| Melanoma | 4.9 | 2.5 | 8.3 | 102 |

Data are reported as average of FPKM (number Fragments Per Kilobase of exon per Million reads) and were generated by The Cancer Genome Atlas (https://cancergenome.nih.gov/). The number of analyzed samples for each tumor type is reported.

Vascular expression of CD93 and MMRN2 was scored in tissue microarrays containing duplicate tissue cores per sample (1 mm diameter) of 455 samples of 15 distinct human tumors. Immunohistochemical staining was performed as previously described using the following antibodies: anti-MMRN2 (HPA020741, Atlas Antibodies) and anti-CD93 (HPA009300 and HPA012368, Atlas Antibodies). Since CD93 and MMRN2 were heterogeneously expressed in tumor vessel, a semiquantitative scoring method was used based on the proportion of positive vessels in each core. The frequency of positively stained vessels was scored in a blinded fashion on a scale from 0 to 3 (0=no vessels stained, 1=minority of vessels stained, 2=majority of vessels stained, 3=strong staining in the majority of vessels).

### Statistical Analysis

In this study, the data analyses were performed with the Sigma-Plot GraphPad Prism 6 software (Graphpad Software) and the values represent the mean±SD obtained from at least three measurements on randomized samples. The Pearson product-moment correlation coefficient (r) was used to estimate association between two variables. All P values reported were two-tailed and P<0.05 was considered statistically significant.

### Results

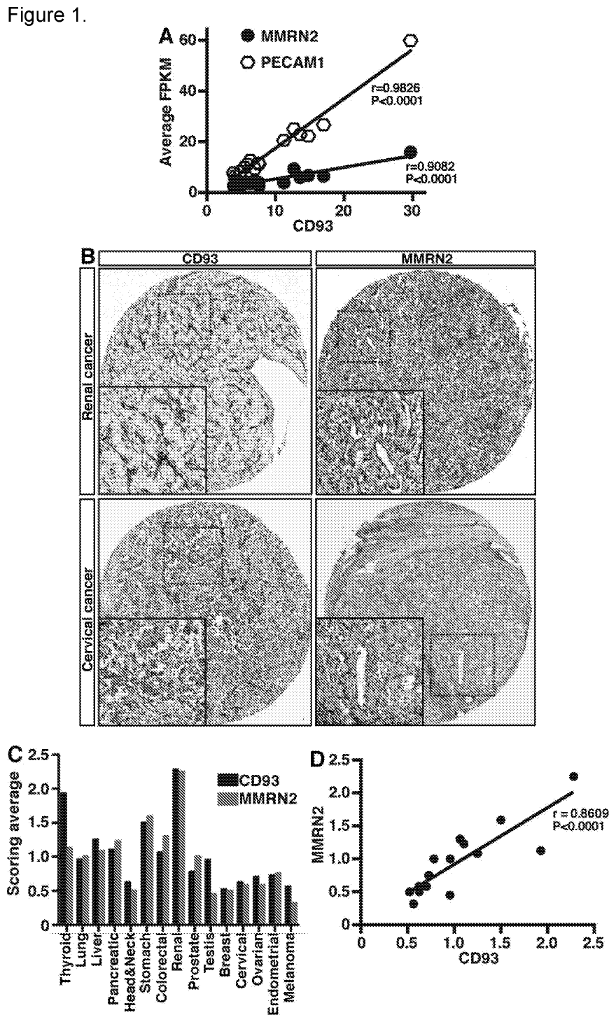

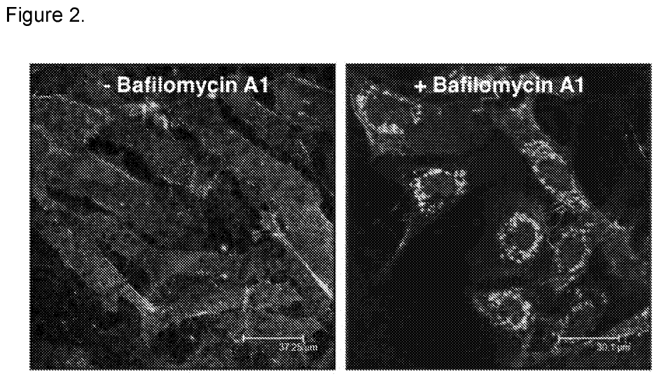

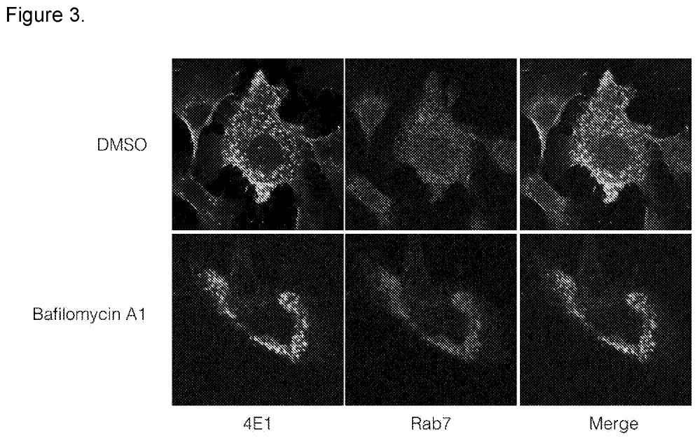

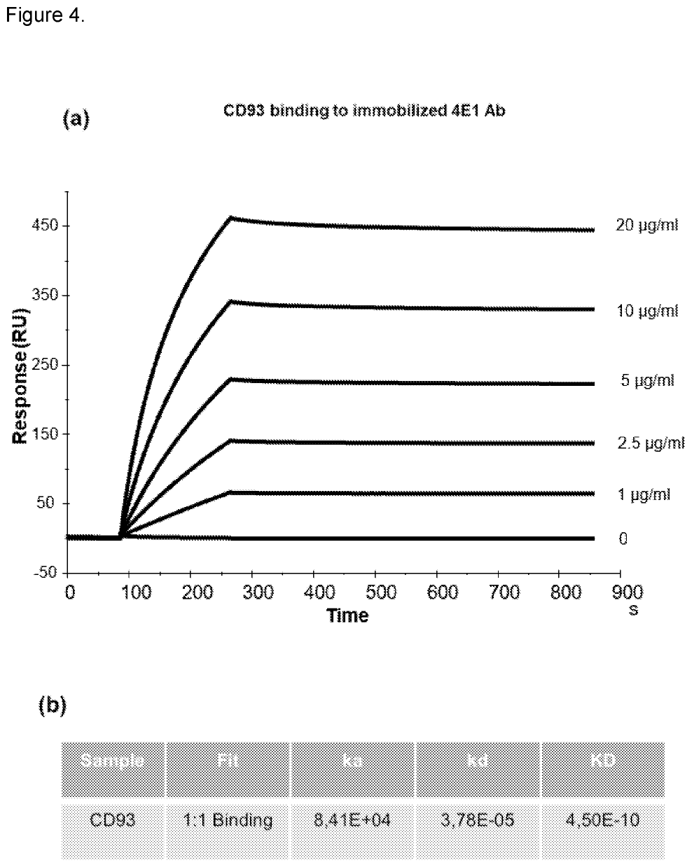

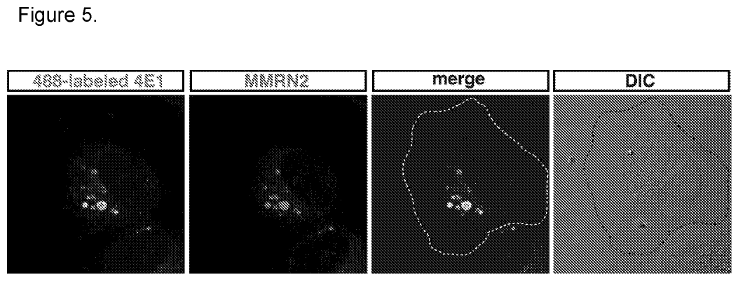

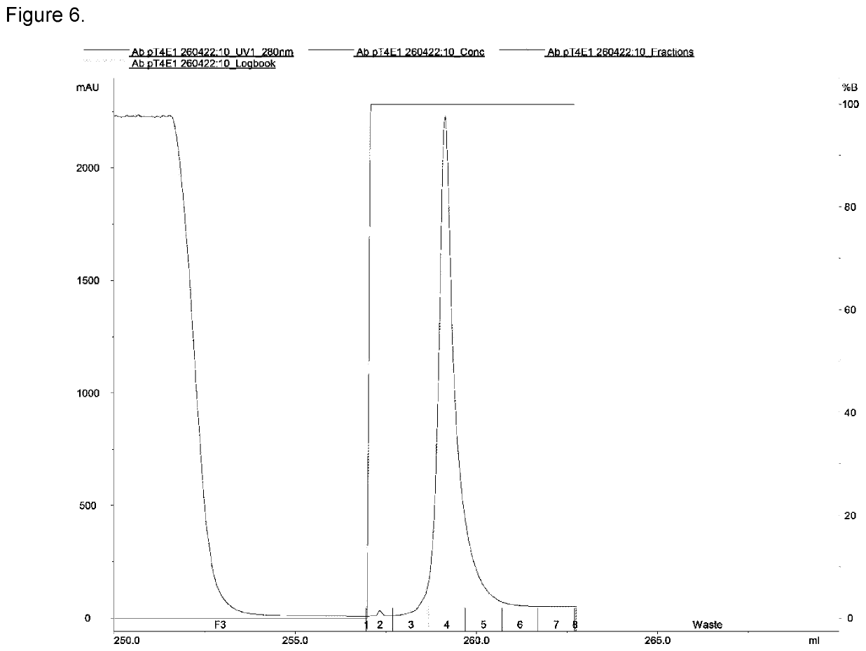

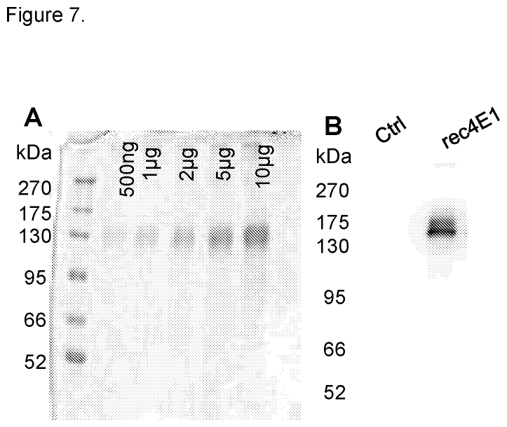

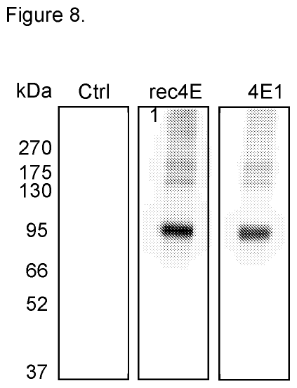

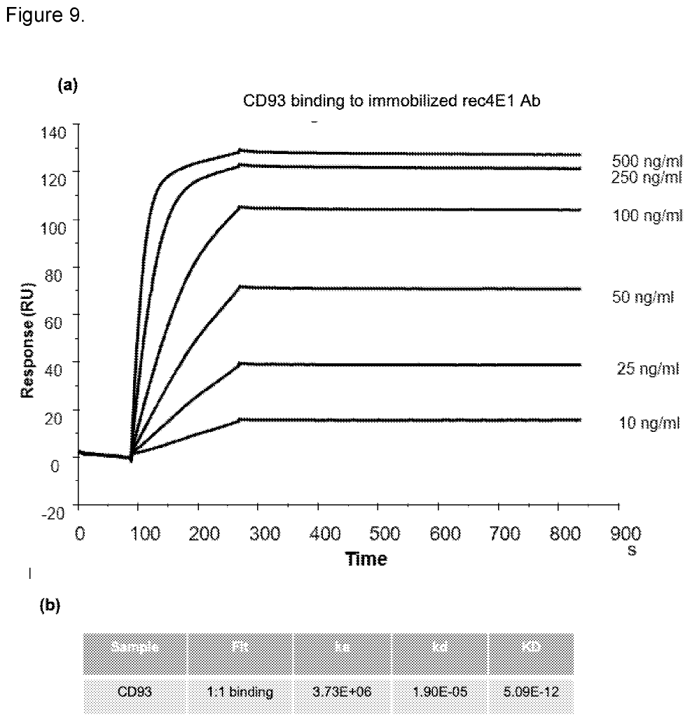

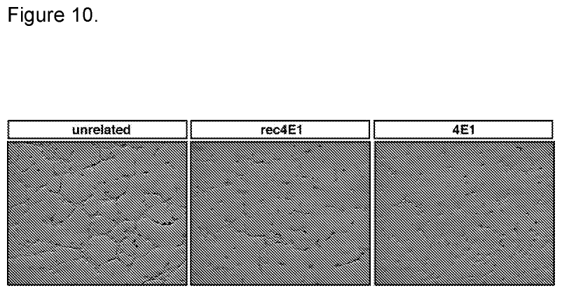

It has been shown that the vascular expression of CD93 is upregulated in some human tumor samples (15, 16, 21). Also, MMRN2, the binding partner of CD93, was found consistently deposited along human tumor blood vessels (17,18). To determine whether there is a correlation between CD93 and MMRN2 expression in tumor vasculature, we first compared CD93, MMRN2, and PECAM-1 (an EC marker) RNA-seq data collected from 15 human cancer types: thyroid, lung, liver, pancreatic, head and neck, stomach, colorectal, renal, prostate, testis, breast, cervical, ovarian, endometrial, and melanoma (see Table 1 for details). These analyses showed a strong correlation between similar expression levels of CD93 and MMRN2 and the degree of tumor vascularization, estimated through PECAM-1 expression (FIG. 1A). Next, we analyzed the fraction of blood vessels positive for CD93 and MMRN2 in tumor tissue microarrays (FIG. 1B), containing a total of 455 samples of tissues from 15 different human tumors, and calculated the scoring average for each cancer type (FIG. 1C). When correlating the scoring of CD93 staining with the scoring of MMRN2 staining in the same tissue microarray cores, we found that high levels of CD93 corresponded to high levels of MMRN2 and vice versa (FIG. 1D). Altogether, these data indicate that CD93 and MMRN2 are coexpressed in tumor vessels and that the levels of these proteins are increased in ipervascularized tumors, suggesting a functional role of the CD93-MMRN2 interaction in tumor angiogenesis progressive development.

### Example 2: Monoclonal Antibody 4E1 Purification and Uptake Tests

### Materials and Methods

### Cells

Primary human umbilical vein endothelial cells (HUVECs) were obtained from LONZA and authenticated by FACS analysis with an anti-CD31 mAb and throughout the culture by assessment of typical morphology by the investigators. Mycoplasma-negative cultures were ensured by weekly tests. Cells were cultured in EGMTM-2 Endothelial Cell Growth Medium-2 (LONZA) and maintained in a humidified atmosphere at 37° C. and 5% CO₂.

### Monoclonal Antibody

Female BALB/c mice were fourfold injected with 8×10⁶ cultured HUVEC released from the substrate by using detaching buffer (10 mM EDTA in PBS) and resuspended as single cells in PBS. Immune spleen cells were fused with X63-Ag8 myeloma cells. 1021 hybridomas were screened for their ability to produce mAbs that bind endothelial cell surface antigens by flow cytometry. Briefly, after non-enzymatic detachment, HUVECs were incubated in PBS containing 5% fetal bovine serum to block unspecific binding and labeled with primary antibodies diluted in PBS containing 1% bovine serum albumin (BSA). Staining was revealed using anti-mouse conjugated secondary antibodies. Next, positive mAbs were selected for their ability to not recognize antigens expressed on the surface of human epithelial HEK-293 cells. Data acquisition and analysis were performed in a Becton Dickinson FACS Calibur flow cytometer using CellQuest software. 61 different antibodies that recognized endothelial surface molecules were purified by affinity chromatography with HiTrap Protein G columns (GE Healthcare) and used in inhibition assays.

The cDNA sequence of the immunoglobulin genes of the 4E1 monoclonal antibody was determined and is shown above (light chain: SEQ ID No: 3 and heavy chain: SEQ ID No: 4).

Moreover, the protein sequences of the heavy and light chain (VDJ or VJ domain, without constant domain) of 4E1 were determined (SEQ ID No: 2 and SEQ ID No: 1) and the CDR sequences of 4E1 according to the Chothia nomenclature deduced.

The CDR Sequences of 4E1 are: KASQSVDYDGDSYMN (LCDR1; SEQ ID No: 5), AASNLES (LCDR2; SEQ ID No: 6), QQNNEDPRT (LCDR3; SEQ ID No: 7), GYTFASY (HCDR1; SEQ ID No: 8), YPGNGD (HCDR2; SEQ ID No: 9), and LDWYFDL (HCDR3; SEQ ID No: 10).

### mAb 4E1 is of the IgG1 k Chain Isotype.

Mouse IgG Isotype Control mAbs (#10400C) were obtained from Thermo Fisher Scientific.

### Antibody Uptake Assays

20,000 HUVECs were seeded onto gelatin-coated glass coverslips, grown for 24 hours in EGMTM-2 medium, and subsequently incubated 18 h with 10 μg/ml 4E1 in the presence or not of 100 nM Bafilomycin A1 (Sigma-Aldrich). Next, cells were fixed in 3% paraformaldehyde, permeabilized with 0.1% Triton X-100 in PBS, and blocked with 0.3% w/v BSA in PBS, each for 15 min at room temperature. Subsequently, cells were washed twice with PBS and incubated 1 h at 4° C. with a secondary Alexa Fluor 488-labeled anti-mouse IgG (Thermo Fisher Scientific), diluted at 25 μg/mL in PBS/0.1% Tween-20, then incubated 15 min with ToPro-3 stain (cat. #T3605; ThermoFisher Scientific) diluted 1:1,000 (1 μM) in PBS to label nuclei. Finally, samples were washed three times with PBS/0.1% Tween-20 pH 7.2 and coverslips were mounted with Mowiol mounting medium and fluorescence images were acquired. For anti-Rab7 co-staining, permeabilized cells were incubated 1.5 h with rabbit anti-Rab7 (D95F2) mAb (Cat. #9367) diluted 1:100 in PBS/0.1% Tween-20. The secondary antibody used for Rab-7 staining was conjugated with Alexa Fluor-568 (Thermo Fisher Scientific).

### Confocal Laser Scanning Microscopy

Fluorescent images were captured using a Leica TCS SP2 AOBS confocal laser-scanning microscope and overlaid images were produced. A Leica HCX PL APO Ibd.BL 63×/1.40 oil objective was used. Fluorochromes and fluorescent proteins were excited at the optimal wavelength ranging from 458 nm to 633 nm and images (512×512 resolution) acquired at a scan speed of 400 Hz image lines/sec. Confocal scanner configuration was set as follows: pinhole at 1.0 Airy diameter and line averaging function at 4.

### Results

The mAb 4E1 is internalized via the lysosomal pathway.

We carried out internalization assays in HUVECs using 4E1 and a secondary Alexa Fluor-488 conjugated antibody as outlined in the Materials & Methods section above. Bafilomycin A1, a specific inhibitor of vacuolar-type proton pump, which inhibits acidification and protein degradation in lysosomes and promotes perinuclear distribution of lysosomes, induced perinuclear accumulation of 4E1 signal, suggesting that 4E1 is internalized via the lysosomal pathway (FIG. 2). To confirm this hypothesis, we stained Bafilomycin-treated and untreated HUVECs for 4E1 and Rab7, a well-known marker of late endosomes, that controls aggregation and fusion of late endocytic structures/lysosomes. As shown in FIG. 3, in Bafilomycin-treated cells, 4E1 accumulated in Rab7-labeled structures, confirming that the mAb 4E1 is internalized via the lysosomal pathway.

### Example 3: Binding Analysis of the 4E1 Monoclonal Antibody

### Materials and Methods

### Cell Lines

HUVEC cells were grown as described in Example 2.

### Monoclonal Antibody

4E1 monoclonal antibody was obtained as described in Example 2. Human Lenti-X 293T cells (Takara Bio Inc) were cultured using standard conditions.

### Production of the CD93 Recombinant Protein

The chimeric construct containing the extracellular domain of CD93 fused to a 6×His tag was generated by PCR amplification of a cDNA clone corresponding to the complete sequence of the human gene, using the following primers: forward 5′GAGAGGATCCGACACGGAGGCGGTGG3′ (SEQ ID NO:11) and reverse 5′GAGAGAATTCTCAGTGGTGATGGTGATGATGCTTTTGCCCGTCAGTGC′ (SEQ ID NO:12). The PCR fragment was cloned into the pCS2 vector (Addgene). The construct was confirmed by sequencing. To obtain soluble CD93 protein, human Lenti-X 293T cells were transiently transfected using lipofectamine 2000 (Thermo Fisher Scientific) according to the manufacturer's instructions. 24 h after transfection, the cells were rinsed with PBS and grown in DMEM without supplements for 16 h. The recombinant CD93 was purified by nickel-affinity chromatography in a AKTA purifier (GE Healthcare), using a step-gradient elution protocol relying on imidazole as a competitive agent. Fractions containing CD93, identified by Western Blot analysis, were pooled and dialyzed in PBS. Protein concentration was determined by spectrophotometric analysis.

### Surface Plasmon Resonance (SPR) Equilibrium Analysis and Binding Constants

The binding analysis was performed using a BIAcore 3000 equipped with a CM5 sensor chip. Briefly, 4E1 antibody was immobilized via amine groups on a CM5 sensor chip following standard procedures (flow rate 5 μl/min). For the immobilization, 4E1 diluted in 10 mM Na acetate pH 4.0 at the concentration of 10 μg/ml was injected for 360 sec over the dextran matrix on flow cell, which had been previously activated with a mixture of EDC-NHS for 420 sec. After injection of the antibody, a pulse of Ethanolamine 1M was performed to neutralize activated groups. A reference flow cell to be used as blank, was simultaneously subjected to identical procedures, but the injection of Antibody, which was substituted with injection of dilution buffer alone (HBS-EP+). Recombinant purified CD93 protein was diluted in HBS-EP+ at different concentration (1 μg/ml, 2.5 μg/ml, 5 g/ml and 20 μg/ml) and injected for 180 s at a flow rate of 60 μl/min on flow cell 4 and on reference flow cell 3. Dissociation was followed for 600 sec, regeneration was achieved with a short pulse of glycine 10 mM pH2.0. Sensorgrams illustrated in the figure are obtained by subtraction from sensorgrams on Fc4 of those obtained on Fc3. Kinetic rates were calculated applying a 1:1 binding as fitting model using the Bia T200 evaluation software 2.0.1

### Results

By SPR equilibrium analysis, it was found that 4E1 exhibits an affinity of KD (M)=4.50*10⁻¹⁰ for CD93, and a kinetic association (ka) and dissociation (kd) rates of 8.41*10⁴ and 3.78*10⁻⁵, respectively (FIG. 4).

### Example 4: 4E1 Monoclonal Antibody Labeling and Immunofluorescence Assays

### Materials and Methods

### Cells

HUVECs were grown as described in Example 2.

Antibody Labeling with Fluorochrome

The 4E1 monoclonal antibody was obtained as described in Example 2.

As indicated in Example 2, the protein sequence of the heavy and light chain (VDJ or VJ domain, without constant domain), respectively, of 4E1 was determined (SEQ ID No: 2 and SEQ ID No: 1). Moreover, the CDR sequences of 4E1 according to the Chothia nomenclature were determined. The CDR Sequences of 4E1 are: KASQSVDYDGDSYMN (LCDR1; SEQ ID No: 5), AASNLES (LCDR2; SEQ ID No: 6), QQNNEDPRT (LCDR3; SEQ ID No: 7), GYTFASY (HCDR1; SEQ ID No: 8), YPGNGD (HCDR2; SEQ ID No: 9), and LDWYFDL (HCDR3; SEQ ID No: 10). mAb 4E1 is of the IgG1 k chain isotype.

Zip Alexa Fluor Rapid Antibody Labeling kit (Thermo Fisher Scientific) was used to efficiently and quickly label the mAb 4E1 with Alexa Fluor 488 following the manufacturer's instructions. Briefly, 10 μl of 1 M sodium bicarbonate solution were added to 100 μl of 4E1 solution (1 mg/ml). Next, the antibody solution was incubated for 15 min at room temperature with the fluorochrome solution (Component B) and used in immunofluorescence experiments.

### Immunofluorescence

Cells were seeded onto gelatin-coated glass coverslips and fixed in 3% paraformaldehyde for 15 min at room temperature during the late spreading phase. Samples were permeabilized with 0.1% Triton X-100 in PBS, and blocked with 0.3% w/v BSA in PBS, each for 15 min at room temperature. Next, cells were washed with PBS and incubated 1 h at room temperature with Alexa Fluor 488-labeled 4E1 and rabbit anti-MMRN2 antibodies. Then, samples were washed three times with PBS/0.1% Tween-20 and incubated with secondary Alexa Fluor 568-labeled anti-rabbit antibodies (Thermo Fisher Scientific), diluted at 25 μg/mL in PBS/0.1% Tween-20. After washing, coverslips were mounted with Mowiol mounting medium. Fluorescent images were captured using a Leica TCS SP2 AOBS confocal laser-scanning microscope and overlaid images were produced. A Leica HCX PL APO Ibd.BL 63×/1.40 oil objective was used. Fluorochromes and fluorescent proteins were excited at the optimal wavelength ranging from 458 nm to 633 nm and images (512×512 resolution) acquired at a scan speed of 400 Hz image lines/sec. Confocal scanner configuration was set as follows: pinhole at 1.0 Airy diameter and line averaging function at 4.

### Results

To assess whether the mAb 4E1 could be linked to a fluorescent dye, we performed a conjugation reaction using a commercial kit, which employs Alexa Fluor 488 as dye. Since in spreading cells CD93 is associated with its ligand MMRN2 in internalized vesicles [12], we carried out immunofluorescence experiments on late spreading HUVECs to verify whether conjugated 4E1 retained its binding activities by staining endocytic vesicles. The use of 488-labeled 4E1 and anti-MMRN2 antibodies clearly showed that CD93 colocalized with MMRN2 in internalized vesicles, demonstrating that 4E1, after a conjugation event, retains its binding peculiarity to CD93 (FIG. 5).

### Example 5: Generation of the Recombinant Antibody rec4E1

### Materials and Methods

### Cell Culture, RNA Extraction, and Sequencing

4E1 antibody-secreting hybridoma cells were cultured in RPMI containing 10% FBS and 10 ng/mL human IL-6 (OriGene). Total cellular RNA was extracted from hybridoma cells using the EuroGold Trifast reagent (Euroclone) following manufacturer's instructions. Cloning and sequencing of the purified RNA was purchased from Applied Biological Materials (abm). The protein sequences of the heavy and light chain (VH and VL) of 4E1 were determined and the CDR sequences of 4E1 according to the Chothia nomenclature deduced. The CDR Sequences of 4E1 are: KASQSVDYDGDSYMN (LCDR1; SEQ ID No: 5), AASNLES (LCDR2; SEQ ID No: 6), QQNNEDPRT (LCDR3; SEQ ID No: 7), GYTFASY (HCDR1; SEQ ID No: 8), YPGNGD (HCDR2; SEQ ID No: 9), and LDWYFDL (HCDR3; SEQ ID No: 10).

### DNA Cloning

The VH-4e1 and VL-4e1 constructs, containing the VH and VL sequences of 4E1 respectively, optimized for CHO cell expression, and cloned into pBluescript vector, were purchased from GenScript. Recombinant vector for high yield production of recombinant 4E1 (rec4E1) in CHO cells was constructed in two steps. First, the VH sequence from the V_{H}-4e1 plasmid was subcloned into the AgeI/AfeI sites of the pTRIOZ vector (InvivoGen) leading to the recombinant vector pT-VH4e1. Second, the VL sequence was PCR amplified from the VL-4e1 plasmid and assembled into the PmeI/PshAI sites of pT-V_{H}4e1 using the NEBuilder HiFi DNA assembly cloning kit (New England Biolabs), leading to the vector pT-VHL4e1 able to express rec4E1.

### Results

The VH and VL regions of the monoclonal antibody 4E1 were sequenced and subcloned into the pTRIOZ vector, which has been designed specifically for high yield production of whole recombinant monoclonal antibodies. The pTRIOZ plasmid contains three distinct cassettes for the expression of the H and L chains of an antibody as well as antibiotic selection with Zeocin in both bacterial and mammalian cells. For the precise expression ratio of the H and L chains of an antibody, necessary for successful antibody secretion, the H and L chains are under the control of the human ferritin heavy and light chain promoters respectively, which natively drive the co-expression of the two ferritin subunits. Additionally, pTRIOZ expresses the constant region of the H chain from murine IgG1 with the T252M mutation, which increases the antibody affinity for Protein A and facilitates purification steps. Thus, the pT-VHL4e1 plasmid we generated is able to express an IgG1 with the variable regions of 4E1, to perform stable gene expression via Zeocin selection in mammalian cells, and to obtain high yield purified protein using Protein A.

### Example 6: Expression and Purification of rec4E1

### Materials and Methods

### Cell Culture and Transfections

HEK 293 and CHO cells were grown in DMEM supplemented with 10% FBS. Transient transfection experiments were performed using the Transporter 5® Transfection Reagent (Polysciences), following manufacturer's instructions. CHO transfectants were selected in DMEM supplemented with 10% FBS and Zeocin (InvivoGen) at 400 μg/mL.

### Protein Purification and Immunoblotting Analysis

Protein purification was performed using a fast protein liquid chromatograph (FPLC) Akta purifier system endowed of a UV-900 Detector and a Frac-900 fraction collector (GE Healthcare). Protein A-affinity chromatography was carried out in a HiTrap rProtein A FF 1 mL column (Cytiva). Ab-containing cell supernatant from CHO cells was centrifuged, filtered through a 0.45 μm filter, and diluted with an equal volume of binding buffer (0.02 M sodium phosphate, 3 M NaCl, pH 7.0). 0.1 M sodium citrate pH 3 was used as elution buffer. Eluted fractions were neutralized with Tris-HCl 1 M pH 8.5, dialyzed in PBS, and concentrated with Centricon filters (Merck KGaA). Protein concentration was measured using a NanoPhotometer (Implen).

Proteins were separated by SDS-PAGE and transferred to a PVDF membrane. Equal loading was confirmed by staining in Ponceau S solution. Filters were blocked for 1 h at room temperature in PBS containing 5% dry milk and 0.1% Tween-20, followed by an overnight incubation at 4° C. with rec4E1 or HRP-conjugated goat anti-mouse antibodies. Filters were washed and finally developed using enhanced chemiluminescence.

### Results

To produce high yield of rec4E1, CHO cells were transfected with the plasmid pT-VHL4e1 and selected in Zeocin-containing culture medium for 10 days. Cell supernatant was collected and subjected to affinity-chromatography using Protein A (FIG. 6). To check recombinant antibody expression and the degree of contaminant-free purification, eluted fractions were pooled, dialyzed, concentrated, and analyzed by SDS-PAGE and Western blot analysis, which showed the specific purification of rec4E1 (FIG. 7). Next, to investigate whether the purified rec4E1 was able to recognize human CD93, HEK 293 cells were transfected with a construct expressing human CD93. Western blot analysis of cell lysates under non-reducing conditions showed that rec4E1 equally well bound the conformational epitope exposed on the surface of the human CD93 protein as 4E1 obtained from hybridoma cells (FIG. 8), suggesting that rec4E1 holds the same binding properties as 4E1.

### Example 7: Binding Analysis of rec4E1

### Materials and Methods

### Cell Culture and Transfections

HEK 293 cells were grown and transfected as described in Example 2.

### Production of the CD93 Recombinant Protein

The chimeric construct containing the extracellular domain of CD93 fused to a 6×His tag was generated by PCR amplification of a cDNA clone corresponding to the complete sequence of the human gene, using the following primers: forward 5′GAGAGGATCCGACACGGAGGCGGTGG3′ (SEQ ID NO:11) and reverse 5′GAGAGAATTCTCAGTGGTGATGGTGATGATGCTTTTGCCCGTCAGTGC3′ (SEQ ID NO:12). The PCR fragment was cloned into the pCS2 vector (Addgene). The construct was confirmed by sequencing. To obtain soluble CD93 protein, HEK 293 cells were transiently transfected. 24 h after transfection, the cells were rinsed with PBS and grown in DMEM without supplements for 16 h. The recombinant CD93 was purified by nickel-affinity chromatography in an AKTA purifier system (GE Healthcare), using a step-gradient elution protocol relying on imidazole as a competitive agent. Fractions containing CD93, identified by Western Blot analysis, were pooled and dialyzed in PBS. Protein concentration was determined by spectrophotometric analysis.

### Surface Plasmon Resonance (SPR) Equilibrium Analysis and Binding Constants

The binding analysis was performed using a BIAcore 3000 equipped with a CM5 sensor chip. Briefly, rec4E1 or 4E1 antibodies were immobilized via amine groups on a CM5 sensor chip following standard procedures. For the immobilization, the antibody diluted in 10 mM Na acetate pH 4.0 at the concentration of 10 μg/ml was injected for 360 sec over the dextran matrix on flow cell, which had been previously activated with a mixture of EDC-NHS for 420 sec. After injection of the antibody, a pulse of Ethanolamine 1 M was used to neutralize activated groups. A reference flow cell to be used as blank was simultaneously subjected to identical procedures, but the antibody was substituted with dilution buffer (HBS-EP+). Recombinant purified CD93 protein was diluted in HBS-EP+ at different concentration (10, 25, 50, 100, 250, and 500 ng/mL) and injected for 180 s at a flow rate of 60 μl/min on all flow cells. Dissociation was followed for 600 sec and regeneration was achieved with a short pulse of 10 mM glycine pH 2.0. Kinetic rates were calculated applying a 1:1 binding as fitting model using the Bia T200 evaluation software 2.0.1.

### Results

By SPR equilibrium analysis, it was found that rec4E1 exhibits an affinity of KD (M)=5.09*10⁻¹² for CD93, and a kinetic association (ka) and dissociation (kd) rates of 3.73*10⁶ and 1.90*10⁻⁵, respectively (FIG. 9), suggesting that rec4E1 shows a higher affinity for CD93 than mAb 4E1.

### Example 8: Functional Analysis of rec4E1

### Materials and Methods

### Cell Culture

Primary human umbilical vein endothelial cells (HUVECs) were obtained from LONZA and authenticated by FACS analysis with an anti-CD31 antibody and throughout the culture by assessment of typical morphology by the investigators. Mycoplasma-negative cultures were ensured by weekly tests. Cells were cultured in EBM-2 Endothelial Cell Growth Medium-2 (LONZA) and maintained in a humidified atmosphere at 37° C. and 5% CO₂.

### Tube Formation Assay

Formation of capillary-like tube structures was examined on Matrigel (growth factor reduced, BD Biosciences), in which was incorporated unrelated antibodies, 4E1, or rec4E1 at the concentration of 500 nM. 0.1 mL of Matrigel was polymerized on 24-well plates and 2×10⁴ HUVECs detached from the culture plate with a non-enzymatic method were seeded in complete growth medium. Cells were grown for 6 h in complete medium in the presence of 500 nM unrelated antibodies, 4E1, or rec4E1. After 8 h, images of tube formation were captured using an inverted microscope (Leica DMIL) equipped with a Nikon DXM1200 digital camera.

### Results

Matrigel is a substrate that allows attachment and differentiation of endothelial cells. In the presence of unrelated antibodies, HUVECs formed a complete network of tubular-like structures, whereas the treatment with rec4E1 abrogated the cells' capability to form tubules on Matrigel. The same impaired tubulogenesis was observed for HUVECs grown on Matrigel in the presence of the CD93-neutralizing antibody 4E1 (FIG. 10), suggesting that rec4E1 and 4E1 play the same inhibitory role against CD93.

From the above description and the above-noted examples, the advantage attained by the rec4E1 monoclonal antibody described and obtained according to the present invention are apparent.

### REFERENCES

1. Folkman J. Angiogenesis in cancer, vascular, rheumatoid and other disease. Nat Med. 1995 January; 1 (1): 27-31.
2. Garner, A., Vascular diseases, in: Pathobiology of ocular disease. A dynamic approach, Garner, A., and Klintworth, G. K. (eds.), 2nd edition, Marcel Dekker, New York (1994), pp. 1625-1710.
3. Galluzzi L, Vacchelli E, Fridman W H, Galon J, Sautes-Fridman C, Tartour E, Zucman-Rossi J, Zitvogel L, Kroemer G: Trial Watch: Monoclonal antibodies in cancer therapy. Oncoimmunology 2012, 1 (1): 28-37.
4. Khanna S, Komati R, Eichenbaum D A, et al Current and upcoming anti-VEGF therapies and dosing strategies for the treatment of neovascular AMD: a comparative review BMJ Open Ophthalmology 2019; 4: e000398.
5. Mammadzada P, Corredoira P M, Andrè H. The role of hypoxia-inducible factors in neovascular age-related macular degeneration: a gene therapy perspective. Cell Mol Life Sci 2020, 77:819-833.
6. Bucci C, Thomsen P, Nicoziani P, McCarthy J, van Deurs B. Rab7: a key to lysosome biogenesis. Mol Biol Cell. 2000 February; 11 (2): 467-80.
7. Orlandini M, Galvagni F, Bardelli M, Rocchigiani M, Lentucci C, Anselmi F, Zippo A, Bini L, Oliviero S. The characterization of a novel monoclonal antibody against CD93 unveils a new antiangiogenic target. Oncotarget. 2014 May 15; 5 (9): 2750-60.
8. Epitope Mapping, A practical approach, Oxford University Press 2001, Editors: Olwyn Westwood and Frank Hay.
9. Tosi G M, Neri G, Barbera S, Mundo L, Parolini B, Lazzi S, Lugano R, Poletto E, Leoncini L, Pertile G, Mongiat M, Dimberg A, Galvagni F, Orlandini M: The binding of CD93 to Multimerin-2 promotes choroidal neovascularization. Investigative ophthalmology & visual science 2020, 61 (8): 30.
10. Galvagni F, Nardi F, Spiga O, Trezza A, Tarticchio G, Pellicani R, Andreuzzi E, Caldi E, Toti P, Tosi G M, Santucci A, Iozzo R V, Mongiat M, Orlandini M. Dissecting the CD93-Multimerin 2 interaction involved in cell adhesion and migration of the activated endothelium. Matrix Biol. 2017 December; 64:112-127.
11. Winter, G., Milstein, C. Man-made antibodies. Nature 349, 293-299 (1991).
12. Barbera S, Nardi F, Elia I, Realini G, Lugano R, Santucci A, Tosi G M, Dimberg A, Galvagni F, Orlandini M. Cell Comm Signal 2019 17:55.

What is claimed is:

1. An isolated recombinant anti-CD93 antibody, wherein said antibody comprises:
a variable domain of a light chain (VJ) having the amino acid sequence of SEQ ID NO:1;
and
a variable domain of a heavy chain (VDJ) having the amino acid sequence of SEQ ID NO:2.

2. The isolated recombinant anti-CD93 antibody of claim 1, wherein said antibody comprises six CDR regions, said six CDR regions being:
a CDR-L1 having the amino acid sequence of SEQ ID NO:5;
a CDR-L2 having the amino acid sequence of SEQ ID NO:6;
a CDR-L3 having the amino acid sequence of SEQ ID NO:7;
a CDR-H1 having the amino acid sequence of SEQ ID NO:8;
a CDR-H2 having the amino acid sequence of SEQ ID NO:9; and
a CDR-H3 having the amino acid sequence of SEQ ID NO:10.

3. The isolated recombinant anti-CD93 antibody of claim 1, wherein the antibody is monoclonal, is humanized or human.

4. The isolated recombinant anti-CD93 antibody of claim 1, wherein the antibody is a diabody, a triabody or a tetrabody.

5. The isolated recombinant anti-CD93 antibody of claim 1, wherein the antibody is a full-length monoclonal antibody and optionally a bispecific antibody.

6. A polynucleotide encoding the isolated recombinant anti-CD93 antibody of claim 1.

7. A vector comprising the polynucleotide of claim 6, wherein the vector is optionally an expression vector.

8. A host cell comprising the vector of claim 7, wherein the host cell is prokaryotic, eukaryotic, or mammalian.

9. A method for producing the isolated recombinant anti-CD93 antibody of claim 1, said method comprising (a) expressing a vector comprising a polynucleotide encoding the isolated recombinant anti-CD93 antibody in a suitable host cell, and (b) recovering the antibody.

10. A pharmaceutical composition comprising (i) the isolated recombinant anti-CD93 antibody of claim 1 or (ii) a polynucleotide encoding the isolated recombinant anti-CD93 antibody, wherein the pharmaceutical composition optionally further comprises a carrier.

11. The pharmaceutical composition of claim 10, wherein said isolated recombinant anti-CD93 antibody is linked to a therapeutically active substance.

12. The pharmaceutical composition of claim 11, wherein the pharmaceutical composition is suitable for oral, parenteral, topical, rectal, intravenous, subcutaneous, intramuscular, intranasal, intravaginal, through the oral mucosa, the lung mucosa, or transocular administration, and wherein said pharmaceutical composition is administered incorporated into liposomes, microvescicles, bound to molecular carriers or combined with molecules selected from the group consisting of molecules that allow temporary opening of the blood-brain barrier, anti-inflammatory molecules, monoclonal antibodies and drugs with immunosuppressive activity.

13. (canceled)

14. A method of treating an angiogenic disorder, the method comprising administering to a subject in need thereof the isolated recombinant anti-CD93 antibody of claim 1 or a pharmaceutical composition comprising the isolated recombinant anti-CD93 antibody.

15. The method of claim 14, wherein the angiogenic disorder is a retinopathy selected from the group consisting of Age-related macular degeneration (AMD), diabetic retinopathy, intraocular neovascular syndromes, and proliferative retinopathies.

16. A method of treating rheumatoid arthritis or psoriasis, the method comprising administering to a subject in need thereof the isolated recombinant anti-CD93 antibody of claim 1 or a pharmaceutical composition comprising the isolated recombinant anti-CD93 antibody.

17. A method of treating a tumor, the method comprising administering to a subject in need thereof the isolated recombinant anti-CD93 antibody of claim 1 or a pharmaceutical composition comprising the isolated recombinant anti-CD93 antibody.

18. The method of claim 17, wherein the tumor is an epithelial tumor and/or is selected from the group consisting of astrocytoma, oligodendroglioma, meningioma, neurofibroma, glioblastoma, ependymoma, Schwannoma, neurofibrosarcoma, medulloblastoma, melanoma, pancreatic cancer, prostate carcinoma, head and neck cancer, breast cancer, lung cancer, ovarian cancer, endometrial cancer, renal cancer, neuroblastoma, squamous carcinoma, hepatoma, colon cancer, mesothelioma and epidermoid carcinoma.

19. The isolated recombinant anti-CD93 antibody of claim 1, wherein said antibody is linked to a diagnostic compound selected from the group consisting of a radionuclide, a chemoluminescent compound, a fluorescent compound, a dye, and an enzyme.

20. The isolated recombinant anti-CD93 antibody of claim 1, wherein the antibody is used as an in vitro diagnostic agent or as an in vitro biotechnological agent.

21. The pharmaceutical composition of claim 11, wherein the therapeutically active substance is a chemotherapeutic compound selected from an alkylating agent, an antineoplastic antibiotic, an antimetabolite, and a natural source derivative, a cytotoxic compound, a cytostatic compound, a cytokine, a nanoparticle, and a radionuclide.


SEQUENCE LISTING (12 sequences)

SEQ ID NO: 1 - protein, 111 aa, Homo sapiens
  REGION 1..111: note = light chain (VJ domain) of 4E1
DIVLTQSPTSLAVSLGQRATISCKASQSVDYDGDSYMNWYQQKPGQPPRLLIYAASNLESGIPARFSGSGSGTDFTLNIHPVEEEDAATYYCQQNNEDPRTFGGGTKLEIK

SEQ ID NO: 2 - protein, 115 aa, Homo sapiens
  REGION 1..115: note = heavy chain (VDJ domain) of 4E1
QEQLQQPGTELVKPGASVRLSCKTSGYTFASYTLHWVKQTPGQGLDWIGAIYPGNGDTSYNQKFKDKATLTADKSSSTAYMQLSNLTSEDSAVYYCARLDWYFDLWGAGTTVTVS

SEQ ID NO: 3 - DNA, 396 bp, Homo sapiens
  misc_feature 1..396: note = light chain
  source 1..396: mol_type = other DNA
atggagtttcagacccaatcctgctatgggtgctcctgctctgggttccaggctccactggtgacattgtgctgacccaatctccaacttctttggctgtgtctctagggcagagggccaccatctcctgcaaggccagccaaagtgttgattatgatggtgatagttatatgaactggtatcaacagaaaccaggacagccacccagactcctcatctatgctgcatccaatctagaatctgggatcccagccaggtttagtggcagtgggtctgggacagacttcaccctcaacatccatcctgtggaggaggaggatgctgcaacctattactgtcagcaaaataatgaggatcctcggacgttcggtggaggcaccaagttggaaataaaac

SEQ ID NO: 4 - DNA, 406 bp, Homo sapiens
  misc_feature 1..406: note = Heavy chain
  source 1..406: mol_type = other DNA
atgggatggagctgtatcatcctcttcttggtagcaacagctacaggtgtccactcccaggagcaactgcagcagcctgggactgagctggtgaagcctggggcctcagtgaggctgtcctgcaagacttctggctacacatttgccagttacactttgcactgggtaaagcagacacctggacagggcctggactggattggagctatttatccaggaaatggtgatacttcctacaatcagaaattcaaagacaaggccacattaactgcagacaaatcttccagcacagcctatatgcagctcagtaacctgacatctgaggactctgcggtctattactgtgcaaggttagactggtacttcgatctctggggcgcagggaccacggtcactgtctctgcag

SEQ ID NO: 5 - protein, 15 aa, Homo sapiens
  REGION 1..15: note = CDR-L1 (or LCDR1)
KASQSVDYDGDSYMN

SEQ ID NO: 6 - protein, 7 aa, Homo sapiens
  REGION 1..7: note = CDR-L2 (or LCDR2)
AASNLES

SEQ ID NO: 7 - protein, 9 aa, Homo sapiens
  REGION 1..9: note = CDR-L3 (or LCDR3)
QQNNEDPRT

SEQ ID NO: 8 - protein, 7 aa, Homo sapiens
  REGION 1..7: note = CDR-H1 (or HCDR1)
GYTFASY

SEQ ID NO: 9 - protein, 6 aa, Homo sapiens
  REGION 1..6: note = CDR-H2 (or HCDR2)
YPGNGD

SEQ ID NO: 10 - protein, 7 aa, Homo sapiens
  REGION 1..7: note = CDR-H3 (or HCDR3)
LDWYFDL

SEQ ID NO: 11 - DNA, 26 bp, synthetic construct
  misc_feature 1..26: note = Forward primer
  source 1..26: mol_type = other DNA
gagaggatccgacacggaggcggtgg

SEQ ID NO: 12 - DNA, 48 bp, synthetic construct
  misc_feature 1..48: note = Reverse primer
  source 1..48: mol_type = other DNA
gagagaattctcagtggtgatggtgatgatgcttttgcccgtcagtgc